(19) United States
(12) Patent Application Publication
(10) Pub. No.: US 2004/0093039 A1
(43) Pub. Date: May 13, 2004

(54) Gastrointestinal pacemaker

(76) Inventor: Raphael Schumert, Sherman Oaks, CA (US)

Correspondence Address: David A. Farah, M.D., SHELDON & MAK PC, 9th Floor, 225 South Lake Avenue, Pasadena, CA, 91101, US

(21) Appl. No.: 10/692,915

(22) Filed: Oct. 24, 2003

Related U.S. Application Data
(60) Provisional application No. 60/421,418, filed on Oct. 25, 2002.

(51) Int. Cl. A61N 1/08

(52) U.S. Cl. 607/40

(57) ABSTRACT

A gastrointestinal pacemaker device for pacing the gastrointestinal tract wall of a patient. A method for affecting peristalsis or for treating a disruption of peristalsis. An anchoring device for a gastrointestinal pacemaker.

### CROSS-REFERENCE TO RELATED APPLICATIONS

The present Application claims the benefit of U.S. patent application Ser. No. 60/421,418 titled “Gastrointestinal Pacemaker Capsule” and filed Oct. 25, 2002, the contents of which are incorporated in this disclosure by reference in their entirety.

### BACKGROUND

Gastrointestinal contents are normally propagated through the gastrointestinal tract by involuntary contractions of the muscle tissue in the gastrointestinal tract wall, originating in the proximal esophagus and extending through the distal colon. These muscle contractions, referred to as “peristalsis” or “peristaltic waves,” are disrupted by a large number of diseases and conditions, such as for example, diabetic gastroparesis, intestinal pseudo-obstruction, nausea of pregnancy, constipation, dumping syndrome, irritable bowel syndrome and postoperative ileus. Among other problems, disruptions of peristalsis can cause aspiration, bloating, constipation, diarrhea, malnutrition, nausea, pain, vomiting and wound dehiscence.

Current treatments for disruptions of peristalsis include nutritional support while the gastrointestinal tract recovers its normal peristalsis, pharmacotherapy directed to the cause of the peristalsis disruption, and pharmacotherapy directed to stimulating or inhibiting peristalsis. While useful, each of these treatments is associated with disadvantages. For example, nutritional support while the gastrointestinal tract recovers can require an invasive procedure and generally increases the length of hospitalization. Pharmacotherapy directed to the cause of the peristalsis disruption, such as antibiotic therapy directed against gastrointestinal pathogens, can take days to restore normal peristalsis. Similarly, pharmacotherapy directed to stimulating or inhibiting peristalsis can be difficult to administer orally due to the disruption of peristalsis, and can also take days to restore normal peristalsis.

Therefore, it would be useful to have another method for treating disruptions of peristalsis. Further, it would be useful to a device for treating disruptions of peristalsis.

### SUMMARY

According to one embodiment of the present invention, there is provided a gastrointestinal pacemaker device for pacing the gastrointestinal tract wall of a patient. The gastrointestinal pacemaker comprises a) an outer housing configured to be introduced into the gastrointestinal tract of a patient; b) an inner cavity within the outer housing; c) a pulse generator in the inner cavity for generating intermittent discharges to the gastrointestinal tract wall of the patient; and d) a power source connected to the pulse generator.

In one embodiment, the gastrointestinal pacemaker further comprises one or more than one electrode connected to the pulse generator, where the one or more than one electrode delivers the discharges from the pulse generator to the gastrointestinal tract wall. In another embodiment, the power source is a self-contained battery. In another embodiment, the power source is configured to receive energy from an external source. In another embodiment, the power source comprises a piezoelectric crystal. In another embodiment, the power source generates electrical activity by converting intrinsic gastrointestinal mechanical or physical activity to electrical power.

In a preferred embodiment, the gastrointestinal pacemaker further comprises a) a sensor for sensing gastrointestinal tract wall electrical activity; and b) one or more than one microprocessor connecting the pulse generator to the sensor. In a particularly preferred embodiment, the pulse generator delivers discharges to the gastrointestinal tract wall that have an amplitude, duration, frequency, pattern or pulse width or combination of these parameters determined, at least in part, by the electrical activity of the gastrointestinal tract sensed by the sensor.

In another preferred embodiment, the gastrointestinal pacemaker further comprises one or more than one electrode connected to the sensor, where the one or more than one electrode senses activity of the gastrointestinal tract wall. In another preferred embodiment, the gastrointestinal pacemaker further comprises one or more than one electrode connected to the pulse generator for delivering the discharges from the pulse generator to the gastrointestinal tract wall, and to the sensor for sensing electrical activity of the gastrointestinal tract wall; and further comprises an electrode switching circuit to control the function and polarity of the one or more than one electrode.

In one embodiment, the gastrointestinal pacemaker further comprises a telemetry unit comprising a receiver and a transmitter. In another embodiment, the gastrointestinal pacemaker further comprises random access memory connected to one or more than one microprocessor. In another embodiment, the gastrointestinal pacemaker further comprises a gastrointestinal tract location sensor.

In one embodiment, the gastrointestinal pacemaker where the outer housing is generally elliptical and has an axial length of between about 1.5 cm and 3 cm, and a midline diameter of between about 8 mm and 12 mm. In another embodiment, the gastrointestinal pacemaker of claim 2, where the one or more than one electrode is a plate attached to the outer housing. In another embodiment, the gastrointestinal pacemaker where the one or more than one electrode comprises a coiled flexible wire.

In a preferred embodiment, the gastrointestinal pacemaker further comprises one or more than one anchoring device to attach the gastrointestinal pacemaker to the gastrointestinal tract wall. In one embodiment, at least one of the one or more than one anchoring device comprises a piercing element for piercing the gastrointestinal tract wall. In another embodiment, at least one of the one or more than one anchoring device comprises means for applying suction to the gastrointestinal tract wall. In another embodiment, at least one of the one or more than one anchoring device comprises both a piercing element for piercing the gastrointestinal tract wall and means for applying suction to the gastrointestinal tract wall.

According to another embodiment of the present invention, there is provided a method for affecting peristalsis or treating a disruption of peristalsis. The method comprises a) selecting a patient having a need for altering peristalsis or having a disruption of peristalsis; b) introducing a gastrointestinal pacemaker into the gastrointestinal tract of the patient; and c) activating the gastrointestinal pacemaker to affect peristalsis. In one embodiment, the method further comprises removing the gastrointestinal pacemaker. In one embodiment, the gastrointestinal pacemaker further comprises one or more than one anchoring device; and the method further comprises activating the one or more than one anchoring device thereby anchoring the gastrointestinal pacemaker to the gastrointestinal tract wall. In another embodiment, at least one of the one or more than one anchoring device comprises a piercing element for piercing the gastrointestinal tract wall; and activating the anchoring device comprises piercing the gastrointestinal tract wall with the piercing element. In another embodiment, at least one of the one or more than one anchoring device comprises means for applying suction to the gastrointestinal tract wall; and activating the anchoring device comprises creating suction between the anchoring device and the gastrointestinal tract wall. In another embodiment, at least one of the one or more than one anchoring device comprises both a piercing element for piercing the gastrointestinal tract wall, and means for applying suction to the gastrointestinal tract wall; and activating the anchoring device comprises both piercing the gastrointestinal tract wall with the piercing element and creating suction between the anchoring device and the gastrointestinal tract wall.

In a preferred embodiment, introducing the gastrointestinal pacemaker into the gastrointestinal tract of the patient comprises having the patient swallow the gastrointestinal pacemaker, or placing the gastrointestinal pacemaker through the anus into the large intestine. In a preferred embodiment, introducing the gastrointestinal pacemaker into the gastrointestinal tract of a patient comprises using means for manipulating the gastrointestinal pacemaker. In one embodiment, the method further comprises supplying power to the gastrointestinal pacemaker using a power conduit connected to a power source external to the patient's gastrointestinal tract. In another embodiment, the method further comprises reintroducing the gastrointestinal pacemaker into the gastrointestinal tract of the patient and activating the reintroduced gastrointestinal pacemaker and activating the gastrointestinal pacemaker.

According to another embodiment of the present invention, there is provided an anchoring device for a gastrointestinal pacemaker. The anchoring device comprises a) a housing having a membrane or an opening at the distal end, and containing a piercing element having a proximal end and a distal end, a disk having a proximal side and a distal side, and an elastic structure surrounding the piercing element; b) a space adjoining the proximal side of the disk, where the space is in communication with a pressure producing source; and c) a valve between the pressure producing source and the space; where the proximal end of the piercing element is attached to the distal side of the disk. According to another embodiment of the present invention, there is provided a method of anchoring a gastrointestinal pacemaker. The method comprises a) providing a gastrointestinal pacemaker having an anchoring device according to the present invention; b) introducing the gastrointestinal pacemaker into the gastrointestinal tract of a patient; and c) causing the valve to travel to an activated position, thereby allowing the contents of the pressure producing source to exit the pressure producing source into the space on the proximal side of the disk, compressing the elastic structure and advancing the piercing element through the opening or membrane, and into the gastrointestinal tract wall of the patient. In one embodiment, the method further comprises detaching the anchor from the gastrointestinal tract wall comprising causing the valve to travel to a deactivated position, thereby allowing the elastic structure to force the disk back to its original pre-activation position withdrawing the piercing element from the gastrointestinal tract wall. In another embodiment, the method further comprises detaching the anchor from the gastrointestinal tract wall by detaching the piercing element from the gastrointestinal pacemaker entirely, thereby leaving the piercing element fixed to the gastrointestinal tract wall.

According to another embodiment of the present invention, there is provided an anchoring device for a gastrointestinal pacemaker. The anchoring device comprises a) a housing defining a space, and containing a proximal disk, a distal disk and a rod, connecting the proximal disk to the distal disk; b) a circumferential partition separating the space into a proximal compartment and a distal compartment; c) an elastic structure surrounding the rod between the circumferential partition and the proximal disk; and d) a stop or stops to prevent the proximal disk from advancing distally into the distal compartment; where the proximal compartment comprises a first opening in communication externally and a second opening in communication with a pressure producing source; where the second opening is also in contact externally when the anchoring device is not activated and in contact with the pressure producing source across a valve, during activation; and a conduit in the housing connecting the first opening with the distal compartment.

According to another embodiment of the present invention, there is provided a method of anchoring a gastrointestinal pacemaker. The method comprises a) providing a gastrointestinal pacemaker having an anchoring device according to the present invention; b) introducing the gastrointestinal pacemaker into the gastrointestinal tract of a patient; c) causing the valve to travel to an activated position allowing the contents of the pressure producing source to exit the pressure producing source and communicate with the proximal compartment by the second opening, while closing the communication external to the gastrointestinal pacemaker through the second opening, thereby causing the proximal disk to travel further into the proximal compartment and create suction between the distal disk and the gastrointestinal tract wall in contact with the distal disk. In one embodiment, the method further comprises detaching the anchor from the gastrointestinal tract wall comprising opening a valve in the conduit, thereby breaking suction between the distal disk and the gastrointestinal tract wall, causing the spring to translate the proximal disk to a deactivation position.

According to another embodiment of the present invention, there is provided an anchoring device for a gastrointestinal pacemaker. The anchoring device comprises a) a first section having a piercing element for reversibly piercing the gastrointestinal tract wall of a patient; and b) a second section connected to the first section configured to create suction between the anchoring device and the gastrointestinal tract wall. In one embodiment, the first section comprises a) a housing having a proximal end, and a distal end with an opening communicating externally; where the proximal end of the housing communicates either with a first opening by a conduit, or with the pressure producing source depending on the position of a valve between the conduit and the pressure producing source; and b) a piercing element surrounded by an elastic structure within the housing having a proximal end attached to a disk, and a distal end aligned with the opening in the distal end of the housing. According to another embodiment of the present invention, there is provided a method of anchoring a gastrointestinal pacemaker. The method comprises a) providing a gastrointestinal pacemaker having an anchoring device according to the present invention; b) introducing the gastrointestinal pacemaker into the gastrointestinal tract of a patient; c) creating suction between the second section and the gastrointestinal tract wall; and d) piercing the gastrointestinal tract wall with the piercing element of first section. In a preferred embodiment, the method further comprises detaching the anchor from the gastrointestinal tract wall comprising removing the piercing element from the gastrointestinal tract wall and breaking the suction between the second section and the gastrointestinal tract wall.

### FIGURES

These and other features, aspects and advantages of the present invention will become better understood with regard to the following description, appended claims, and accompanying figures where:

FIG. 1 is a block diagram of some of the components of a gastrointestinal pacemaker according to the present invention;

FIG. 2 is another block diagram of some of the components of a gastrointestinal pacemaker according to the present invention;

FIG. 3 is another block diagram of some of the components of a gastrointestinal pacemaker according to the present invention;

FIG. 4 is another block diagram of some of the components of a gastrointestinal pacemaker according to the present invention;

FIG. 5 shows a partial cutaway, lateral perspective view of a gastrointestinal pacemaker according to one embodiment of the present invention;

FIG. 6 shows a front perspective view of the gastrointestinal pacemaker shown in FIG. 5;

FIG. 7 is a partial, lateral, cross-sectional, perspective view of a gastrointestinal pacemaker according to the present invention, showing one anchoring device according to the present invention before activation;

FIG. 8 is a partial, lateral, cross-sectional, perspective view of a gastrointestinal pacemaker shown in FIG. 7, showing the anchoring device after activation;

FIG. 9 is a partial, lateral, cross-sectional, perspective view of a gastrointestinal pacemaker according to the present invention, showing another anchoring device according to the present invention before activation;

FIG. 10 is a partial, lateral, cross-sectional, perspective view of a gastrointestinal pacemaker shown in FIG. 9, showing the anchoring device after activation;

FIG. 11 is a partial, lateral, cross-sectional, perspective view of a gastrointestinal pacemaker according to the present invention, showing another anchoring device according to the present invention during activation; and

FIG. 12 is a partial, lateral, cross-sectional, perspective view of a gastrointestinal pacemaker shown in FIG. 11, showing the anchoring device after complete activation.

### DESCRIPTION

In one embodiment, there is provided a gastrointestinal pacemaker device for pacing the gastrointestinal tract wall of a patient. In another embodiment, there is provided a method for affecting peristalsis or for treating a disruption of peristalsis. In a preferred embodiment, the method comprises introducing a gastrointestinal pacemaker of the present invention into the gastrointestinal tract, and allowing the gastrointestinal pacemaker to affect peristalsis, thereby countering the disruption of peristalsis. In a preferred embodiment, the method allows for affecting peristalsis or treating a disruption of peristalsis without making an incision, and without delivering pharmacotherapy directed to specifically treating the disruption of peristalsis. In another embodiment, there is provided an anchoring device for a gastrointestinal pacemaker. The anchoring device can comprise a piercing element, means for applying suction or both a piercing element and means for applying suction.

As used in this disclosure, the term “disruptions of peristalsis” and variations of the term, are intended to include one or more than one of 1) an abnormal complete cessation of purposeful peristalsis, 2) an abnormal decrease of peristalsis and 3) an abnormal increase of peristalsis; whether localized, multi-focused or generalized, and whether intermittent or continuous, in the gastrointestinal tract, as will be understood by those with skill in the art with reference to this disclosure.

As used in this disclosure, the term “gastrointestinal” and variations of the term, refer to either the whole alimentary tract, including, for example, the esophagus, stomach, duodenum, small intestine, large intestine and rectum, or to any portion or portions of the alimentary tract, as will be understood by those with skill in the art with reference to this disclosure.

As used in this disclosure, the term “comprise” and variations of the term, such as “comprising” and “comprises,” are not intended to exclude other additives, components, integers or steps.

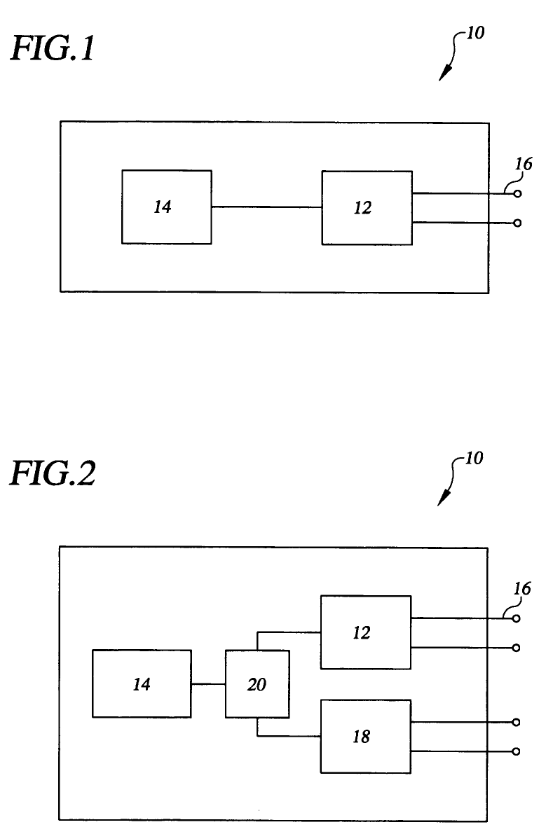

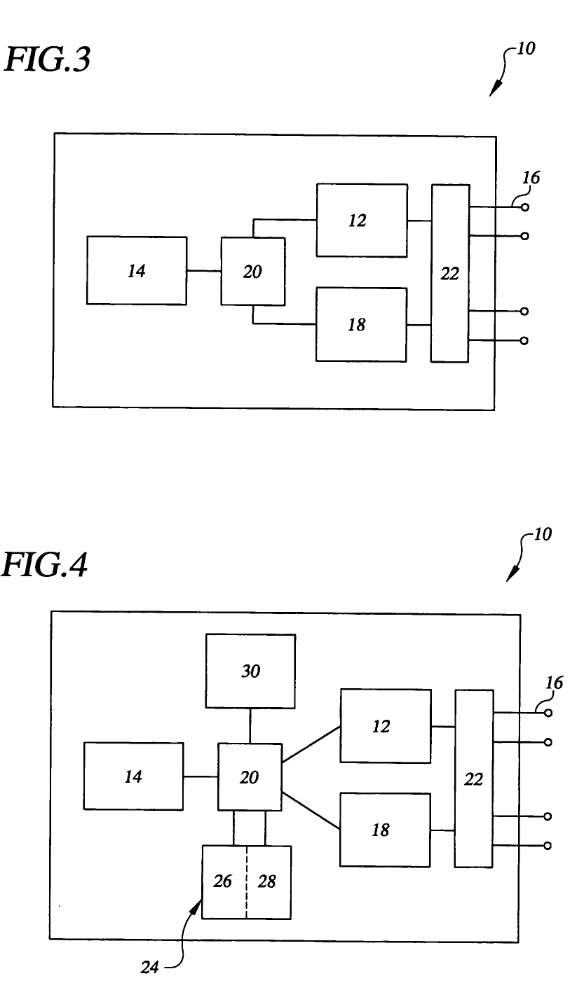

In one embodiment, the present invention is a gastrointestinal pacemaker for affecting peristalsis or treating a disruption of peristalsis. Referring now to FIG. 1, FIG. 2, FIG. 3 and FIG. 4, there are shown by way of example only, block diagrams of some configurations of the possible components of a gastrointestinal pacemaker according to the present invention. As can be seen, the gastrointestinal pacemaker 10 comprises a pulse generator 12 connected to a power source 14. These components of the gastrointestinal pacemaker 10, and other optional components of the gastrointestinal pacemaker 10, will now be disclosed in greater detail.

The gastrointestinal pacemaker 10 of the present invention comprises a pulse generator 12 for generating intermittent discharges, such as for example electrical discharges, and transmitting the discharges to the muscle tissue in the gastrointestinal tract wall. The discharges can amplify, dampen, disrupt, initiate or regulate gastrointestinal electrical activity, or a combination of these, and thereby, gastrointestinal tract wall motility. In a preferred embodiment, the discharges are transmitted through one or more than one electrode 16. In a particularly preferred embodiment, the one or more than one electrode 16 are two electrodes 16 and the discharges are phased between the electrodes 16. Each electrode 16 can be a metal ring or patch on the surface of the pacemaker 10, or can be a flexible wire attached to the surface of the pacemaker 10, or can be any other suitable structure, as will be understood by those with skill in the art with reference to this disclosure. In one embodiment, the distance between the electrode poles is between about 0.5 cm and 2 cm, however any suitable separation can be used, as will be understood by those with skill in the art with reference to this disclosure.

In a preferred embodiment, the pulse generator 12 generates electrical discharges that have an amplitude, duration, frequency, pattern and pulse width, or combination of these parameters, that are programmable, either by other components of the pacemaker 10 or by an external device, in order to treat a specific disruption of peristalsis. In a preferred embodiment, the pulse generator 12 generates electrical discharges that have an amplitude, duration, frequency, pattern or pulse width or combination of these parameters determined, at least in part, by information sensed by a sensor 18, disclosed in more detail below.

The gastrointestinal pacemaker 10 of the present invention further comprises a power source 14. In one embodiment, the power source 14 is a self-contained battery. In a preferred embodiment, the battery is rechargeable. In one embodiment, the power source 14 comprises a piezoelectric crystal attached to a rectifier circuit that can pass direct current to the other components of the gastrointestinal pacemaker 10. In this embodiment, electrical power is derived from mechanical deformation of the crystal by motion of the gastrointestinal tract wall. In another embodiment, the power source 14 is configured to receive energy from an external source, such as by electromagnetic waves, by cell phones, by radiation emitted by another device such as a personal digital assistant, watch or pager. In a preferred embodiment, the external source is radio waves, where the radio waves are received by an antenna connected to the power source 14 and where the power source 14 comprises a radio frequency oscillator and supporting circuitry that enable converting the radio wave energy to direct current power. In another embodiment, the power source 14 generates electrical activity from a piezoelectric crystal within the gastrointestinal pacemaker 10 by converting intrinsic gastrointestinal mechanical or physical activity to electrical power.

In one embodiment, the gastrointestinal pacemaker 10 further comprises a sensor 18 for sensing the intrinsic gastrointestinal tract wall electrical activity, and further comprises a microprocessor 20 connecting the pulse generator 12 to the sensor 18. In a preferred embodiment, the pulse generator 12 delivers discharges to the gastrointestinal tract wall that have an amplitude, duration, frequency, pattern or pulse width or combination of these parameters determined, at least in part, by the electrical activity of the gastrointestinal tract sensed by the sensor 18.

In another preferred embodiment, the sensor 18 senses electrical activity of the gastrointestinal tract through one or more than one electrode 16. In a particularly preferred embodiment, the one or more than one electrode 16 are two electrodes 16 and the sensor 18 senses electrical activity occurring between the electrodes 16. Each electrode 16 can be a metal ring or patch on the surface of the pacemaker 10, or can be a flexible wire attached to the surface of the pacemaker 10, or can be any other suitable structure as will be understood by those with skill in the art with reference to this disclosure.

In another preferred embodiment, the electrodes 16 that the pulse generator 12 discharges through are the same electrodes 16 that the sensor 18 uses to sense the gastrointestinal tract wall electrical activity. In this embodiment, the pacemaker 10 further comprises an electrode switching circuit 22 to control the function and polarity of each electrode 16. Further, the electrode switching circuit 22 determines which of the one or more than one electrode 16 is used by the pulse generator 12 for discharges and which of the one or more than one electrode 16 is used by the sensor 18 for sensing. When being used for discharges, the electrode switching circuit 22 can change the polarity of the one or more than one electrode 16 to create multi-phasic pulses, alternating polarity between pulses, or a series of pulses with different directions of propagation of peristaltic waves, (stimulation vectors). The electrode switching circuit 22 can also compensate to offset internal leakage currents across the switches, involved in sensing. The switching circuitry can also use blanking to prevent stimulation pulses from saturating the sense amplifiers, and can control the one or more than one electrode 16 so that there is at least one bipolar pair to complete an electrical circuit.

In another embodiment, the gastrointestinal pacemaker 10 further comprises a telemetry unit 24 comprising a receiver 26 and a transmitter 28. The telemetry unit 24 communications with external medical devices or programmers, providing input from the sensor 18 and other components of the pacemaker 10, and receiving instructions for the microprocessor 20 and other components of the pacemaker 10, as will be understood by those with skill in the art with reference to this disclosure. The telemetry unit 24 can also communicate with one or more than one other gastrointestinal pacemaker or other device in the gastrointestinal tract, when present.

The microprocessor 20 is an integrated circuit that serves as the information processor for controlling the pacemaker 10 analysis, functions and performance, including the basic timing and routing of the inputs, and the sequencing and parameters of the output. Each of the pulse generator 12, sensor 18, receiver 26 and transmitter 28 can have a separate microprocessor control unit (not shown), or can be controlled solely through the single microprocessor 20 as shown, as will be understood by those with skill in the art with reference to this disclosure. Preferably, the pacemaker 10 further comprises random access memory (RAM) 30, connected to the microprocessor 20, to store data and programming. The RAM 30 stores operating instructions and accumulated device operating and patient data, receives the sensed information about the gastrointestinal activity from sensor 18 through the microprocessor 20, determines if the type of activity according to selected algorithms, and provides the result of the analysis to the microprocessor 20. The microprocessor 20 then activates the pulse generator 12 using the selected programs.

Multiple programs can be stored in the RAM 30 to establish specific profiles for activation of the pulse generator 12. The RAM 30 can also be used to store various parameters that indicate performance of pacemaker 10, and to record the therapies administered, and the responses of the gastrointestinal tract to the therapies.

The microprocessor 20 receives input from the sensor 18, from the telemetry unit 24, and from the RAM 30, when they are present, as well as from any other components of the pacemaker 10. Depending on the programming and the inputs, the microprocessor 20 controls the pulse generator 12, the sensor 18, the electrode switching circuitry 22, the telemetry unit 24 and the RAM 30, when present, as well as from any other components of the pacemaker 10.

In another embodiment, the pacemaker 10 further comprises a gastrointestinal tract location sensor, not shown, to identify the location of the pacemaker 10 in the gastrointestinal tract, as will be understood by those with skill in the art with reference to this disclosure.

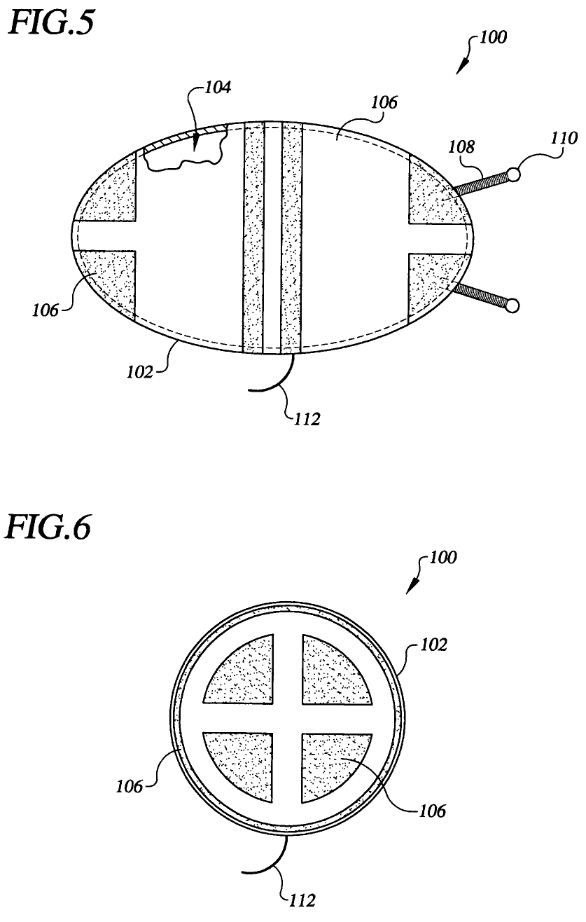

Referring now to FIG. 5 and FIG. 6, there are shown, respectively, a partial cutaway, lateral perspective view of a gastrointestinal pacemaker according to one embodiment of the present invention (FIG. 5), and a front perspective view of the gastrointestinal pacemaker shown in FIG. 5 (FIG. 6). As can be seen the gastrointestinal pacemaker 100 comprises a generally smooth, outer housing 102 and an inner cavity 104 containing the components of the gastrointestinal pacemaker 100. In a preferred embodiment, as shown, the outer housing 102 has a generally ellipsoid shape, though other shapes, both symmetrical and non-symmetrical, could also be used as will be understood by those with skill in the art with reference to this disclosure. In a preferred embodiment, the outer housing 102 comprises a mechanically rigid and stable, biocompatible metal, resin, composite, or a polymer such as ethylene tetrafluoroethylene, though the outer housing 102 can comprise in whole or in part other materials, as will be understood by those with skill in the art with reference to this disclosure. Preferably, the outer housing 102 comprises the minimal wall thickness that is adequate for the necessary rigidity, while permitting the maximum inner cavity 104 space. In one embodiment, the thickness of the outer housing 102 is about 0.5 mm. In another embodiment, the outer housing 102 comprises a plurality of sections, such as two equal sections, that can be separated to replace or service the components of the gastrointestinal pacemaker 100. When in use, the sections of the outer housing 102 are joined and sealed together with an appropriate sealing agent or sealing ring to prevent contact of the components of the gastrointestinal pacemaker 100 with gastrointestinal contents.

By way of example only, suitable dimensions of the outer housing 102 are an axial length of between about 1.5 cm and 3 cm, and a midline diameter of between about 8 mm and 12 mm. These dimensions are configured to allow the gastrointestinal pacemaker 100 to pass completely through the gastrointestinal tract, including through the ileo-cecal valve. Other dimensions, however, are also suitable depending on the components the gastrointestinal pacemaker 100 comprises, the number and distance between the electrodes 106, and the anatomy of the patient being treated.

The gastrointestinal pacemaker 100 further comprises a plurality of bipolar electrodes 106, though other number and placement of the bipolar electrode 106 can also be used, as will be understood by those with skill in the art with reference to this disclosure. In a preferred embodiment, as shown, the electrodes 106 are thin plates attached to the outer surface of the outer housing 102. The plates can be any suitable configuration, such as for example, generally circumferential, circular, helical, or rectangular. The electrodes 106 are made of bio-compatible conductive material, such as for example, iridium platinum, stainless steel or tantalum. Preferably, the electrodes 106 are configured to permit considerable contact with the gastrointestinal tract wall, thereby increasing the amount of information sensed from the gastrointestinal tract wall, such as for example, the directional vector and speed of propagation of peristaltic waves. In one embodiment, as shown, the electrodes 106 are positioned over the front and back tips of the outer housing 102 and also in a circumferential ring over the mid portion of the outer housing 102. This configuration generally enables contact of at least one pair of electrodes 106 with the gastrointestinal tract wall during transit of the gastrointestinal pacemaker 100 through the gastrointestinal tract. However, the placement of the electrodes 106 will vary with the intended use, as will be understood by those with skill in the art with reference to this disclosure. In a preferred embodiment, as shown, the gastrointestinal pacemaker 100 comprises four electrodes 106 in the form of plates at each of the front tip and back tip, where electrodes 106 in opposing quadrants are electrically connected to each other and to one of the bipolar electrodes 106.

In a preferred embodiment, at least two of the electrodes 106 comprise a coiled flexible wire 108 attached to the electrode 106 on the surface of the outer housing 102. Each wire 108 is coated with a bio-compatible, conductive material, such as platinum. Each wire 108 can comprise an expanded distal end 110, such as a plate, or such as a ball as shown. The wires 108 and the expanded distal ends 110 improve conductivity and assist the electrodes 106 in making contact with the walls of the gastrointestinal tract, particularly with the walls of the stomach and large intestine.

The electronic components of the gastrointestinal pacemaker 100 are preferably disposed in the inner cavity 104 on one or more than one miniature printed circuit board assemblies, in a space efficient manner. The semiconductor components are preferably disposed and electrically connected on the sides of the assemblies. As will be understood by those with skill in the art with reference to this disclosure, the circuit board assemblies are preferably multilayer boards having plurality of circuit traces, vias and contact pads to facilitate electrical interconnection of the various terminals of the integrated circuits and any discrete electrical components, such as resistors, capacitors or transistors. Miniature flexible electrical tracing form the electrical interconnection between the multiple printed circuit board assemblies.

In another embodiment, the gastrointestinal pacemaker 100 further comprises one or more than one anchoring device to attach the gastrointestinal pacemaker 100 to the gastrointestinal tract wall for variable periods. The one or more than one anchoring device can be any suitable biocompatible structure, as will be understood by those with skill in the art with reference to this disclosure. The one or more than one anchoring device is preferably positioned adjacent to a pair of electrodes 106, and preferably, a program coordinates activation with sensory information to ensure that the electrodes adjacent to the one or more than one anchoring device are touching the gastrointestinal tract wall, or that the electrodes 106 are causing a contraction of the gastrointestinal tract wall, as necessary to anchor the gastrointestinal pacemaker 100 to the gastrointestinal tract wall.

In one embodiment, the anchoring device 112 comprises a piercing element, such as a curved filament, hook or needle, as shown in FIG. 5 and FIG. 6. In another embodiment, the gastrointestinal pacemaker 100 comprises a plurality of anchoring devices 112. The plurality of anchoring devices can be multiple copies of the same anchoring device, or can be at least two different anchoring devices. The one or more than one anchoring device 112 can be activated to engage the gastrointestinal tract wall at the time of introduction into the gastrointestinal tract. Alternately, the anchoring device 112 can be contained within the inner cavity 104 at the time of introduction in the gastrointestinal tract and the anchoring device 112 can be activated through the outer housing 102 into position for engaging the gastrointestinal tract wall during transit of the gastrointestinal pacemaker 100 through the gastrointestinal tract. In a preferred embodiment, the anchoring device 112 is configured so that the gastrointestinal pacemaker 100 can be reversibly anchored to the gastrointestinal tract wall without the need for external mechanical manipulation, such as by an operator using an endoscope or colonoscope, as will be understood by those with skill in the art with reference to this disclosure.

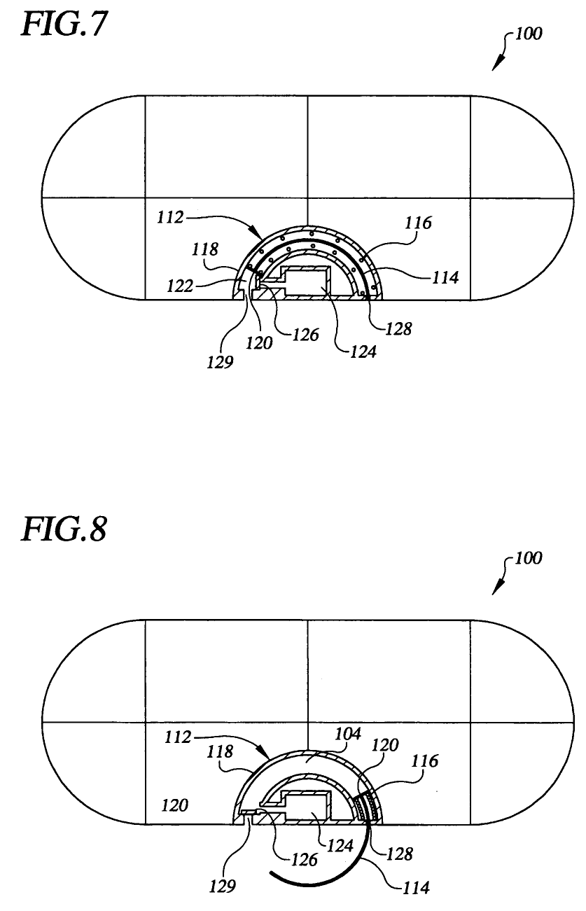

Referring now to FIG. 7 and FIG. 8, there are shown, respectively, is a partial, lateral, cross-sectional, perspective view of a gastrointestinal pacemaker according to the present invention, showing one anchoring device according to the present invention before activation (FIG. 7), and is a partial, lateral, cross-sectional, perspective view of a gastrointestinal pacemaker shown in FIG. 7, showing the anchoring device after activation (FIG. 8). As can be seen, the anchoring device 112 comprises a piercing element 114 surrounded by an elastic structure 116 such as a spring contained within a housing 118 inside the inner cavity 104. The proximal end of the piercing element 114 is attached to the distal side of a disk 120. The opposing proximal side of the disk 120 adjoins a space 122 in communication with a pressure producing source 124 containing pressurized, nontoxic fluid or gas. The anchoring device 112 is activated, as shown in FIG. 8, when the microprocessor, not shown, causes the valve 126 to travel to the activation position, thereby allowing the contents of the pressure producing source 124 to exit the pressure producing source 124 into the space on the proximal side of the disk 120, while closing the communication external to the gastrointestinal pacemaker 100 through the opening 129, compressing the elastic structure 116 or equivalent elastic structure and, thereby, advancing the piercing element 114 through an opening or membrane 128 in the outer housing 102. Alternatively, other activation means can be used to advance the piercing element 114.

In order to detach the gastrointestinal pacemaker 100 from the gastrointestinal tract wall, the valve 126 is returned to its deactivated position allowing the pressurized fluid or gas to communicate external to the outer housing 102 through opening 129. The elastic structure 116 then forces the disk 120 back to its original pre-activation position withdrawing the piercing element 114 from the gastrointestinal tract wall and allowing the gastrointestinal pacemaker 100 to move again within the gastrointestinal tract. Activation of the anchoring device 112 is controlled by the microprocessor, as will be understood by those with skill in the art with reference to this disclosure, and can be done remotely or by a preset program.

In another embodiment, the gastrointestinal pacemaker 100 is detached from the gastrointestinal tract wall by detaching the piercing element 114 from the gastrointestinal pacemaker 100 entirely, thereby leaving the piercing element 114 fixed to the gastrointestinal tract wall permanently or for later retrieval. Detachment of the piercing element 114 can be accomplished by causing the disk 120 with the attached piercing element 114 to be expelled through the outer housing 102 completely.

In a preferred embodiment, the anchoring device 112 also functions as one or more than one of the electrodes 106 of the gastrointestinal pacemaker 100. The electrical connection to each pole on the anchoring device 112 is insulated. Thus, only one piercing element 114 can be used for each bipolar electrode.

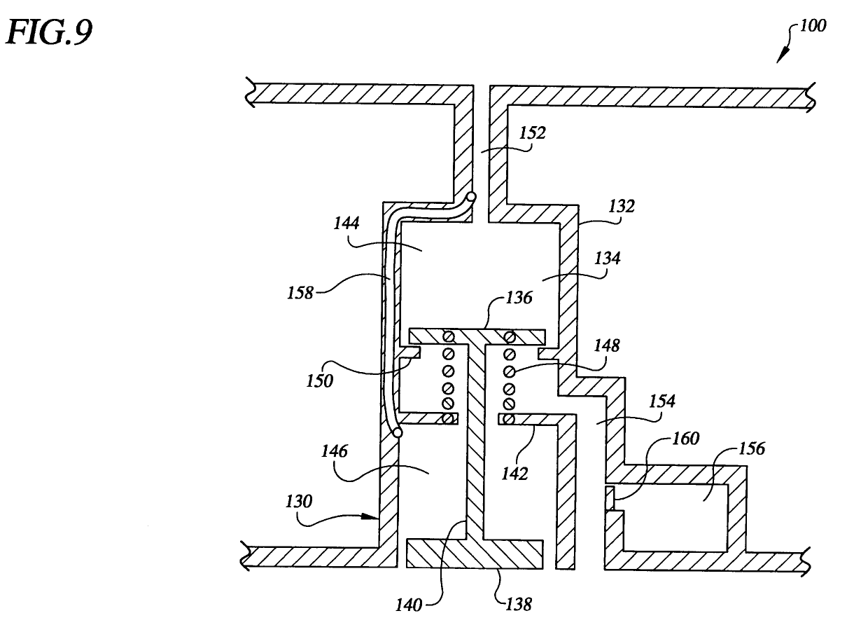

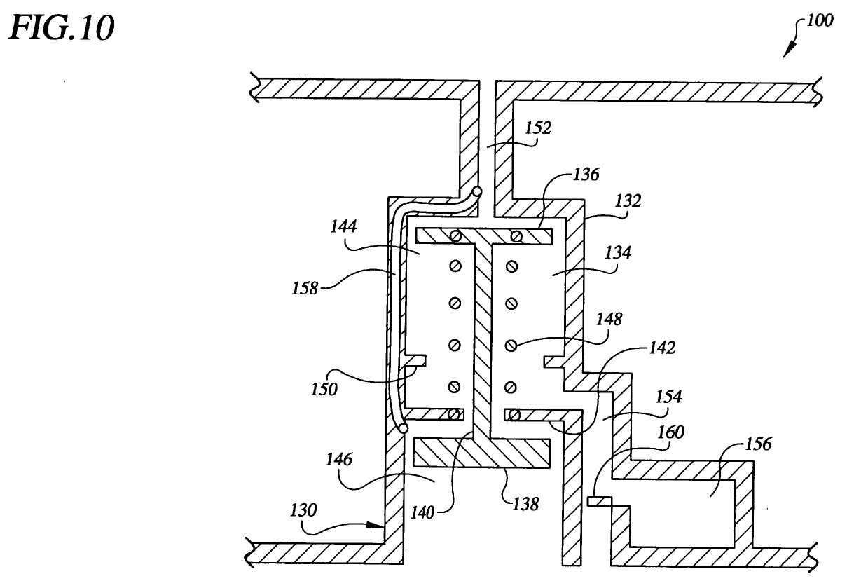

Referring now to FIG. 9 and FIG. 10, there are shown, respectively, is a partial, lateral, cross-sectional, perspective view of a gastrointestinal pacemaker according to the present invention, showing another anchoring device according to the present invention before activation (FIG. 9), and is a partial, lateral, cross-sectional, perspective view of a gastrointestinal pacemaker shown in FIG. 9, showing the anchoring device after activation (FIG. 10). In this embodiment, this anchoring device 130 allows reversible attachment to the gastrointestinal tract wall using suction, rather than using a mechanical piercing element. As can be seen, the anchoring device 130 comprises a housing 132 defining a space 134, a proximal disk 136, a distal disk 138 and a rod 140, connecting the proximal disk 136 to the distal disk 138. The anchoring device 130 further comprises a circumferential partition 142 separating the space 134 into a proximal compartment 144 and a distal compartment 146. The anchoring device 130 further comprises an elastic structure 148, such as a spring, surrounding the rod 140 between the circumferential partition 142 and the proximal disk 136. A stop or stops 150 prevent the proximal disk 136 from advancing distally into the distal compartment 146.

The proximal compartment 144 comprises two openings. The first opening 152 is in communication externally through the outer housing 102. The second opening 154 is in communication with a pressure producing source 156 containing pressurized, nontoxic fluid or gas. The second opening 154 is also further in contact externally through the outer housing 102 when the anchoring device 130 is not activated and in contact with the pressure producing source 156 across a valve 160, during activation. The anchoring device 130 further comprises a conduit 158 in the housing 132 connecting the first opening 152 with the distal compartment 146.

The anchoring device 130 is activated, as shown in FIG. 10, when the microprocessor, not shown, causes the valve 160 to travel to the activation position, thereby allowing the contents of the pressure producing source 156 to exit the pressure producing source 156 and communicate with the proximal compartment 144 by the second opening 154, while closing the communication external to the gastrointestinal pacemaker 100 through the second opening 154. This action causes the proximal disk 136 to travel further into the proximal compartment 144 and creates suction between the distal disk 138 and any surface, such as the gastrointestinal tract wall in contact with the distal disk 138. Deactivation is caused by opening a valve in the conduit 158, thereby equalizing the pressure on both sides of the outer housing 102 and breaking suction between the distal disk 138 and the gastrointestinal tract wall, causing the elastic structure 148 to translate the proximal disk 136 back to the deactivation position. In another embodiment, not shown, the proximal disk 136 and distal disk 138 can be translated to create suction between the distal disk 138 and any surface, such as the gastrointestinal tract wall in contact with the distal disk 138, by introducing a nontoxic gas or liquid in the first opening 152, in combination with a valve to prevent the nontoxic gas or liquid from escaping external to the gastrointestinal pacemaker 100 through the first opening 152.

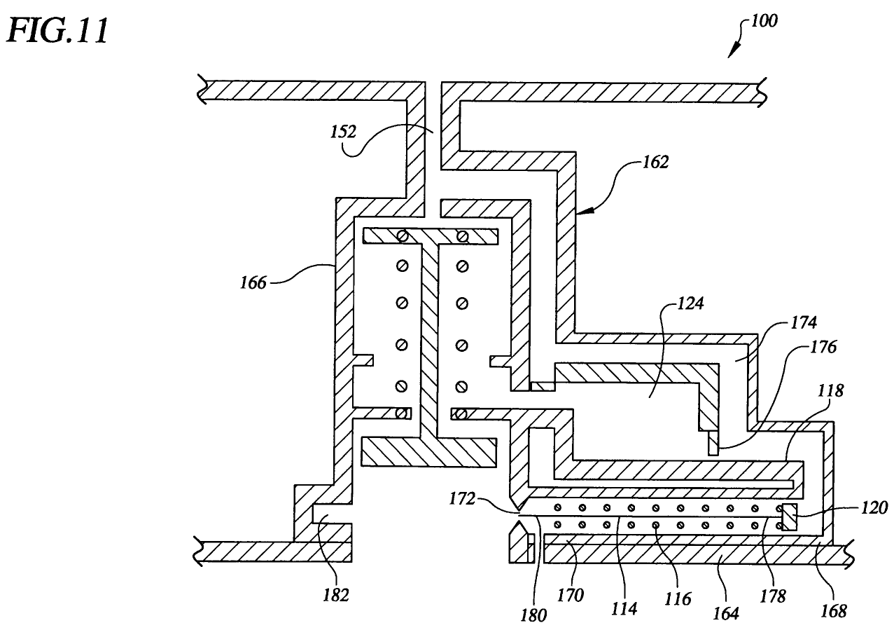

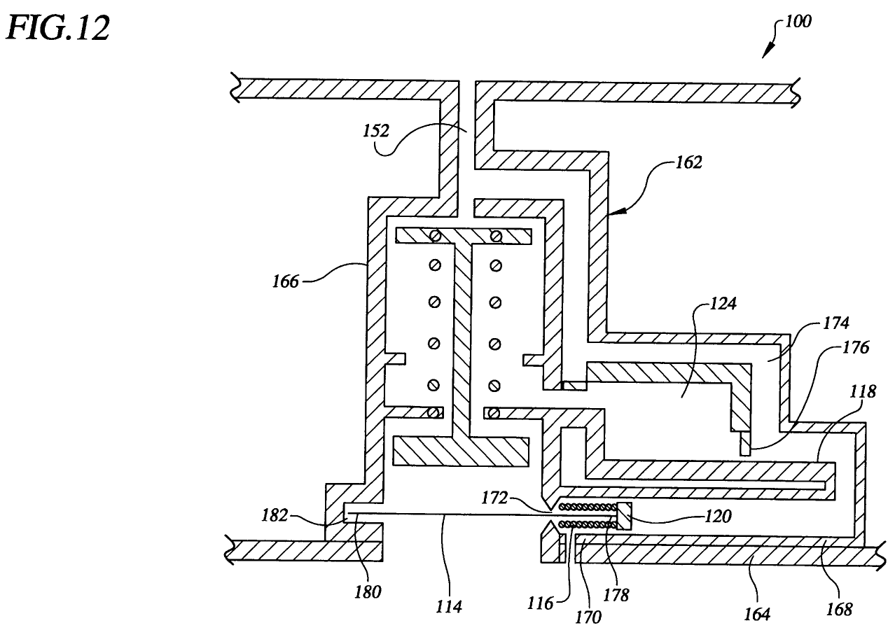

In another embodiment of the present invention, there are provided a gastrointestinal pacemaker 100 comprising an anchoring device 162 that is a combination of the anchoring device 112 and the anchoring device 130. Referring now to FIG. 11 and FIG. 12, there are shown, respectively, is a partial, lateral, cross-sectional, perspective view of a gastrointestinal pacemaker according to the present invention, showing the anchoring device 162 according to the present invention during activation (FIG. 11), and is a partial, lateral, cross-sectional, perspective view of a gastrointestinal pacemaker shown in FIG. 11, showing the anchoring device after complete activation (FIG. 12). As can be seen, the anchoring device 162 comprises two sections, a first section 164 corresponding to the anchoring device 112, and a second section 166 corresponding to the anchoring device 130. Parts of the first section 164 and the second section 166 directly corresponding to parts of the anchoring device 112 and anchoring device 130, discussed above, will be labeled with the same reference numbers.

As can be seen, the first section 164 comprises a housing 118 having a proximal end 168, and a distal end 170 with an opening 172 communicating external to the gastrointestinal pacemaker 100. The proximal end 168 of the housing 118 communicates either with the first opening 152 by a conduit 174, or with the pressure producing source 124 depending on the position of a valve 176. Within the housing 118 is a piercing element 114, such as a needle as shown, having a proximal end 178 attached to a disk 120, and a distal end 180 aligned with the opening 172 in the housing 118. The first section 164 further comprises an elastic structure 116 or equivalent elastic structure surrounding the piercing element 114.

After activation of the second section 166, as disclosed above, creating suction at the surface of the distal disk 138, the valve 176 is moved to the activation position as shown in FIG. 12 causing the disk 120 to travel distally, and thereby, extending the distal end of the piercing element 114 to exit the opening 172 and into the recess 182 in the housing 118. Any tissue of the gastrointestinal tract wall extending into the space 184 due to the suction created by the first section 164 will be penetrated by the piercing element 114, thereby anchoring the gastrointestinal pacemaker 100 to the gastrointestinal tract wall. Deactivation is caused by returning the valve 176 to the deactivation position, and then deactivating the first section 164 as disclosed above, thereby returning the piercing element 114 to the deactivated position and then deactivating the second section 166, thereby releasing the suction from the gastrointestinal tract wall.

According to another embodiment of the present invention there is provided a method for affecting peristalsis or treating a disruption of peristalsis. The method comprises, first, selecting a patient having a need for altering peristalsis or having a disruption of peristalsis. Next, a gastrointestinal pacemaker is introduced into the gastrointestinal tract of the patient. Then, the gastrointestinal pacemaker is activated to affect peristalsis or to treat a disruption of peristalsis. In a preferred embodiment, the gastrointestinal pacemaker is a gastrointestinal pacemaker according to the present invention.

In a preferred embodiment, the gastrointestinal pacemaker is removed after the gastrointestinal pacemaker has affected peristalsis. In a particular preferred embodiment, the gastrointestinal pacemaker is a gastrointestinal pacemaker according to the present invention and removal comprising allowing the gastrointestinal pacemaker to expel itself from the patient's gastrointestinal tract.

In another preferred embodiment, the gastrointestinal pacemaker comprises one or more than one anchoring device and, once the gastrointestinal pacemaker is introduced into the gastrointestinal tract, the one or more than one anchoring device is activated thereby anchoring the gastrointestinal pacemaker to the gastrointestinal tract wall, prior to, during or after activating the gastrointestinal pacemaker to affect peristalsis.

In a preferred embodiment, introducing the gastrointestinal pacemaker into the gastrointestinal tract of the patient comprises having the patient swallow the gastrointestinal pacemaker. In another preferred embodiment, introducing the gastrointestinal pacemaker into the gastrointestinal tract of a patient comprises manually placing the gastrointestinal pacemaker through the anus into the large intestine.

In a preferred embodiment, introducing the gastrointestinal pacemaker into the gastrointestinal tract of a patient comprises using an endoscope or colonoscope or equivalent device in combination with means for manipulating the gastrointestinal pacemaker. The means for manipulating the gastrointestinal pacemaker can comprise a tube or a snare or any suitable structure, as will be understood by those with skill in the art with reference to this disclosure. For example, when a snare is used to introduce the gastrointestinal pacemaker, the gastrointestinal pacemaker housing will preferably have one or more than one circumferential groove configured to allow the snare to grasp the gastrointestinal pacemaker for ease of manipulation. Besides introducing the gastrointestinal pacemaker, the endoscope, colonoscope or equivalent, with or without the snare, can also be used after the gastrointestinal pacemaker is introduced by other means in order to position the gastrointestinal pacemaker for anchoring at a specific location within the gastrointestinal tract.

Activation of the gastrointestinal pacemaker comprises discharging to the gastrointestinal wall in a manner to alter the gastrointestinal wall muscle activity. The type of alteration of the gastrointestinal wall muscle activity will depend upon the disruption of peristalsis being treated, as will be understood by those with skill in the art with reference to this disclosure. By way of example only, where the disruption of peristalsis is an abnormal complete cessation of purposeful peristalsis or an abnormal decrease in peristalsis, such as found in gastroparesis or in constipation, the gastrointestinal pacemaker can pace the gastrointestinal tract wall at a frequency equal to or greater than its natural frequency, such as a frequency that is within about 5 percent to 15 percent greater than the natural frequency, such as approximately 3.3 bpm. Similarly, where the disruption of peristalsis is an abnormal increase in peristalsis, the gastrointestinal pacemaker will pace the gastrointestinal tract wall at a rate which overrides the natural pacing rate for the gastrointestinal tract wall, such as a pacing frequency between about 10 to 400 percent greater than the natural frequency of the gastrointestinal tract wall.

Activation of the gastrointestinal pacemaker can comprise applying discharges to the gastrointestinal tract wall that are a plurality of monophasic, biphasic or triphasic pulses. In one embodiment, the pulse amplitude ranges between 1 mA and 50 mA. In a preferred embodiment, the pulse amplitude is about 5 mA. In one embodiment, the pulse width ranges from about 3 microseconds to about 1000 microseconds. In a preferred embodiment, the pulse width is about 330 microseconds. In one embodiment, the interpulse interval is between about 6 ms and about 600 ms in length. In a preferred embodiment, the interpulse interval is about 60 ms. In one embodiment, the pulse train is between about 1 and 100. In a preferred embodiment, the pulse train is two. However, as will be understood by those with skill in the art with reference to this disclosure, the exact parameters of the discharges will depend on disruption of peristalsis being treated and the portion of the gastrointestinal being treated. Discharges from the gastrointestinal pacemaker can also propel the gastrointestinal pacemaker inside or completely through the gastrointestinal tract, both forward and retrograde.

In another embodiment, the method further comprises supplying power to the gastrointestinal pacemaker using a power conduit connected to a power source external to the patient's gastrointestinal tract. In a preferred embodiment, the power conduit is a snare also used to mechanically manipulate the gastrointestinal pacemaker.

In another embodiment of the present invention, the method further comprises reintroducing the gastrointestinal pacemaker into the gastrointestinal tract of the patient and activating the reintroduced gastrointestinal pacemaker and activating the gastrointestinal pacemaker.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

What is claimed is:

1. A gastrointestinal pacemaker device for pacing the gastrointestinal tract wall of a patient comprising:
a) an outer housing configured to be introduced into the gastrointestinal tract of a patient;
b) an inner cavity within the outer housing;
c) a pulse generator in the inner cavity for generating intermittent discharges to the gastrointestinal tract wall of the patient; and
d) a power source connected to the pulse generator.

2. The gastrointestinal pacemaker of claim 1, further comprising one or more than one electrode connected to the pulse generator, where the one or more than one electrode delivers the discharges from the pulse generator to the gastrointestinal tract wall.

3. The gastrointestinal pacemaker of claim 1, where the power source is a self-contained battery.

4. The gastrointestinal pacemaker of claim 1, where the power source is configured to receive energy from an external source.

5. The gastrointestinal pacemaker of claim 1, where the power source comprises a piezoelectric crystal.

6. The gastrointestinal pacemaker of claim 5, where the power source generates electrical activity by converting intrinsic gastrointestinal mechanical or physical activity to electrical power.

7. The gastrointestinal pacemaker of claim 1, further comprising:
a) a sensor for sensing gastrointestinal tract wall electrical activity; and
b) one or more than one microprocessor connecting the pulse generator to the sensor.

8. The gastrointestinal pacemaker of claim 7, where the pulse generator delivers discharges to the gastrointestinal tract wall that have an amplitude, duration, frequency, pattern or pulse width or combination of these parameters determined, at least in part, by the electrical activity of the gastrointestinal tract sensed by the sensor.

9. The gastrointestinal pacemaker of claim 7, further comprising one or more than one electrode connected to the sensor, where the one or more than one electrode senses activity of the gastrointestinal tract wall.

10. The gastrointestinal pacemaker of claim 7, further comprising one or more than one electrode connected to the pulse generator for delivering the discharges from the pulse generator to the gastrointestinal tract wall, and to the sensor for sensing electrical activity of the gastrointestinal tract wall; and
further comprising an electrode switching circuit to control the function and polarity of the one or more than one electrode.

11. The gastrointestinal pacemaker of claim 1, further comprising a telemetry unit comprising a receiver and a transmitter.

12. The gastrointestinal pacemaker of claim 7, further comprising random access memory connected to one or more than one microprocessor.

13. The gastrointestinal pacemaker of claim 1, further comprising a gastrointestinal tract location sensor.

14. The gastrointestinal pacemaker of claim 1, where the outer housing is generally elliptical and has an axial length of between about 1.5 cm and 3 cm, and a midline diameter of between about 8 mm and 12 mm.

15. The gastrointestinal pacemaker of claim 2, where the one or more than one electrode is a plate attached to the outer housing.

16. The gastrointestinal pacemaker of claim 2, where the one or more than one electrode comprises a coiled flexible wire.

17. The gastrointestinal pacemaker of claim 9, where the one or more than one electrode is a plate attached to the outer housing.

18. The gastrointestinal pacemaker of claim 9, where the one or more than one electrode comprises a coiled flexible wire.

19. The gastrointestinal pacemaker of claim 1, further comprising one or more than one anchoring device to attach the gastrointestinal pacemaker to the gastrointestinal tract wall.

20. The gastrointestinal pacemaker of claim 19, where at least one of the one or more than one anchoring device comprises a piercing element for piercing the gastrointestinal tract wall.

21. The gastrointestinal pacemaker of claim 19, where at least one of the one or more than one anchoring device comprises means for applying suction to the gastrointestinal tract wall.

22. The gastrointestinal pacemaker of claim 19, where at least one of the one or more than one anchoring device comprises both a piercing element for piercing the gastrointestinal tract wall and means for applying suction to the gastrointestinal tract wall.

23. A method for affecting peristalsis or treating a disruption of peristalsis comprising:
a) selecting a patient having a need for altering peristalsis or having a disruption of peristalsis;
b) introducing a gastrointestinal pacemaker into the gastrointestinal tract of the patient; and
c) activating the gastrointestinal pacemaker to affect peristalsis.

24. The method of claim 23, further comprising removing the gastrointestinal pacemaker.

25. The method of claim 23, where the gastrointestinal pacemaker further comprises one or more than one anchoring device; and
where the method further comprises activating the one or more than one anchoring device thereby anchoring the gastrointestinal pacemaker to the gastrointestinal tract wall.

26. The method of claim 23, where at least one of the one or more than one anchoring device comprises a piercing element for piercing the gastrointestinal tract wall; and
where activating the anchoring device comprises piercing the gastrointestinal tract wall with the piercing element.

27. The method of claim 23, where at least one of the one or more than one anchoring device comprises means for applying suction to the gastrointestinal tract wall; and
where activating the anchoring device comprises creating suction between the anchoring device and the gastrointestinal tract wall.

28. The method of claim 23, where at least one of the one or more than one anchoring device comprises both a piercing element for piercing the gastrointestinal tract wall, and means for applying suction to the gastrointestinal tract wall; and
where activating the anchoring device comprises both piercing the gastrointestinal tract wall with the piercing element and creating suction between the anchoring device and the gastrointestinal tract wall.

29. The method of claim 23, where introducing the gastrointestinal pacemaker into the gastrointestinal tract of the patient comprises having the patient swallow the gastrointestinal pacemaker, or placing the gastrointestinal pacemaker through the anus into the large intestine.

30. The method of claim 23, where introducing the gastrointestinal pacemaker into the gastrointestinal tract of a patient comprises using means for manipulating the gastrointestinal pacemaker.

31. The method of claim 23, further comprising supplying power to the gastrointestinal pacemaker using a power conduit connected to a power source external to the patient's gastrointestinal tract.

32. The method of claim 23, further comprising reintroducing the gastrointestinal pacemaker into the gastrointestinal tract of the patient and activating the reintroduced gastrointestinal pacemaker and activating the gastrointestinal pacemaker.

33. An anchoring device for a gastrointestinal pacemaker comprising:
a) a housing having a membrane or an opening at the distal end, and containing a piercing element having a proximal end and a distal end, a disk having a proximal side and a distal side, and an elastic structure surrounding the piercing element;
b) a space adjoining the proximal side of the disk, where the space is in communication with a pressure producing source; and
c) a valve between the pressure producing source and the space;
where the proximal end of the piercing element is attached to the distal side of the disk.

34. A method of anchoring a gastrointestinal pacemaker comprising:
a) providing a gastrointestinal pacemaker having an anchoring device according to claim 33;
b) introducing the gastrointestinal pacemaker into the gastrointestinal tract of a patient; and
c) causing the valve to travel to an activated position, thereby allowing the contents of the pressure producing source to exit the pressure producing source into the space on the proximal side of the disk, compressing the elastic structure and advancing the piercing element through the opening or membrane, and into the gastrointestinal tract wall of the patient.

35. The method of claim 34, further comprising detaching the anchor from the gastrointestinal tract wall comprising causing the valve to travel to a deactivated position, thereby allowing the elastic structure to force the disk back to its original pre-activation position withdrawing the piercing element from the gastrointestinal tract wall.

36. The method of claim 34, further comprising detaching the anchor from the gastrointestinal tract wall by detaching the piercing element from the gastrointestinal pacemaker entirely, thereby leaving the piercing element fixed to the gastrointestinal tract wall.

37. An anchoring device for a gastrointestinal pacemaker comprising:
a) a housing defining a space, and containing a proximal disk, a distal disk and a rod, connecting the proximal disk to the distal disk;
b) a circumferential partition separating the space into a proximal compartment and a distal compartment;
c) an elastic structure surrounding the rod between the circumferential partition and the proximal disk; and
d) a stop or stops to prevent the proximal disk from advancing distally into the distal compartment;
where the proximal compartment comprises a first opening in communication externally and a second opening in communication with a pressure producing source;
where the second opening is also in contact externally when the anchoring device is not activated and in contact with the pressure producing source across a valve, during activation; and a conduit in the housing connecting the first opening with the distal compartment.

38. A method of anchoring a gastrointestinal pacemaker comprising:
a) providing a gastrointestinal pacemaker having an anchoring device according to claim 37;
b) introducing the gastrointestinal pacemaker into the gastrointestinal tract of a patient;
c) causing the valve to travel to an activated position allowing the contents of the pressure producing source to exit the pressure producing source and communicate with the proximal compartment by the second opening, while closing the communication external to the gastrointestinal pacemaker through the second opening, thereby causing the proximal disk to travel further into the proximal compartment and create suction between the distal disk and the gastrointestinal tract wall in contact with the distal disk.

39. The method of claim 38, further comprising detaching the anchor from the gastrointestinal tract wall comprising opening a valve in the conduit, thereby breaking suction between the distal disk and the gastrointestinal tract wall, causing the spring to translate the proximal disk to a deactivation position.

40. An anchoring device for a gastrointestinal pacemaker comprising:
a) a first section having a piercing element for reversibly piercing the gastrointestinal tract wall of a patient; and
b) a second section connected to the first section configured to create suction between the anchoring device and the gastrointestinal tract wall.

41. The anchoring device of claim 40, where the first section comprises:
a) a housing having a proximal end, and a distal end with an opening communicating externally;
where the proximal end of the housing communicates either with a first opening by a conduit, or with the pressure producing source depending on the position of a valve between the conduit and the pressure producing source; and
b) a piercing element surrounded by an elastic structure within the housing having a proximal end attached to a disk, and a distal end aligned with the opening in the distal end of the housing.

42. A method of anchoring a gastrointestinal pacemaker comprising:
a) providing a gastrointestinal pacemaker having an anchoring device according to claim 41;
b) introducing the gastrointestinal pacemaker into the gastrointestinal tract of a patient;
c) creating suction between the second section and the gastrointestinal tract wall; and
d) piercing the gastrointestinal tract wall with the piercing element of first section.

43. The method of claim 42, further comprising detaching the anchor from the gastrointestinal tract wall comprising removing the piercing element from the gastrointestinal tract wall and breaking the suction between the second section and the gastrointestinal tract wall.
